(19) United States
(12) Patent Application Publication
(10) Pub. No.: US 2025/0077530 A1
(43) Pub. Date: Mar. 6, 2025

(54) SYSTEM AND METHOD FOR MANAGING COLLECTION OF DATA BY A DATA MANAGEMENT SYSTEM

(71) Applicant: Dell Products L.P., Round Rock, TX (US)

(72) Inventors: PREM PRADEEP MOTGI, Austin, TX (US); DHARMESH M. PATEL, Round Rock, TX (US); MANPREET SINGH SOKHI, Santa Clara, CA (US)

(21) Appl. No.: 18/458,380

(22) Filed: Aug. 30, 2023

(51) Int. Cl. G06F 16/2457; G10L 15/26

(52) U.S. Cl. CPC G06F 16/24578; G10L 15/26

(57) ABSTRACT

Methods and systems for managing collection of data by a data management system and from data sources are disclosed. To manage collection of data, data management system may limit the types and quantity of data collected for storage in the data management system. Data management system may prioritize collection of data based on relevancy of the data for one or more purposes with respect to an individual. To identify relevant data, data management system may analyze data, including audio recordings of interactions between the individual for which the data is regarding and other individuals that provide services to the individual, and identify topics of the data. Based on the analysis of the data and identified topics, data management system may establish a ranking order of the topics that are more relevant to the individual.

### FIELD

Embodiments disclosed herein relate generally to data collection management. More particularly, embodiments disclosed herein relate to systems and methods to manage collection of data by a data management system.

### BACKGROUND

Computing devices may provide computer-implemented services. The computer-implemented services may be used by users of the computing devices and/or devices operably connected to the computing devices. The computer-implemented services may be performed with hardware components such as processors, memory modules, storage devices, and communication devices. The operation of these components and the components of other devices may impact the performance of the computer-implemented services.

### BRIEF DESCRIPTION OF THE DRAWINGS

Embodiments disclosed herein are illustrated by way of example and not limitation in the figures of the accompanying drawings in which like references indicate similar elements.

FIG. 1 shows a block diagram illustrating a system in accordance with an embodiment.

FIGS. 2A-2C show diagrams illustrating data flows in accordance with an embodiment.

FIG. 3 show flow diagram illustrating a method of managing collection of data in accordance with an embodiment.

FIG. 4 shows a block diagram illustrating a data processing system in accordance with an embodiment.

### DETAILED DESCRIPTION

Various embodiments will be described with reference to details discussed below, and the accompanying drawings will illustrate the various embodiments. The following description and drawings are illustrative and are not to be construed as limiting. Numerous specific details are described to provide a thorough understanding of various embodiments. However, in certain instances, well-known or conventional details are not described in order to provide a concise discussion of embodiments disclosed herein.

Reference in the specification to “one embodiment” or “an embodiment” means that a particular feature, structure, or characteristic described in conjunction with the embodiment can be included in at least one embodiment. The appearances of the phrases “in one embodiment” and “an embodiment” in various places in the specification do not necessarily all refer to the same embodiment.

References to an “operable connection” or “operably connected” means that a particular device is able to communicate with one or more other devices. The devices themselves may be directly connected to one another or may be indirectly connected to one another through any number of intermediary devices, such as in a network topology.

In general, embodiments disclosed herein relate to methods and systems for managing collection of data by a data management system and from data sources. The data sources may collect and distribute data to the data management system which may receive, store, and/or otherwise manage the data on behalf of an individual. The data may be usable, for example, by (i) an individual for which the data is regarding, and (ii) other individuals to assist the individual. For example, the data may include medical information for an individual and the data may be usable by other individuals such as healthcare providers to diagnose and/or treat the individual for various medical conditions.

However, storing data in the data management system may consume limited storage resources available to the data management system (and/or the data management system may include insufficient resources to store all data collected for an individual).

To manage limited storage resources, the data management system may manage the types of data being collected by data source(s) for storage in the data management system. Some portions of data may include data that is more relevant or helpful for an individual and/or other individuals than other portions of data. Thus, collection of data that may include irrelevant information may be disadvantageous for the individual by reducing the ability of the desired services to be provided and reducing the available storage resources of the data management system.

To address the potential collection of irrelevant data, the data management system may prioritize collection of data on the basis of relevancy of the type of data for one or more purposes with respect to an individual. To discriminate more relevant data from less relevant data, the data management system may analyze data being collected and stored, audio recordings of interactions between the individual and other individuals that provide services, and/or other types of data that may include information identifying relevant content to the individual for which the data is stored.

By identifying relevant content to the individual, the system may establish topics that are relevant to the individual and prioritize collection and storage of data including and/or relating to the topics over collection and storage of data relating to other topics. The data management system may update the topics relevant to the individual and/or adjust relevancy rankings of the topics as new information regarding the topics is obtained. By proactively updating the relevant topics and relevancy rankings for the topics, the data management system may be more likely to collect and store the more desirable data to the individual for which the data is regarding.

Thus, embodiments disclosed herein may provide an improved system for managing collection of data by a data management system and from data sources. The improved data collection system may discriminate more relevant data from less relevant data based on topics relevant to the individual for which the data is being stored. Relevancy ratings of the topics relevant to the individual may be adjusted dynamically as new information is obtained by the system. By doing so, a system in accordance with embodiments disclosed herein may prioritize collection of data based on the relevancy of the data for one or more purposes with respect to the individual. By managing collection of data based on relevancy of the data to the individual, the data collection system may automatically and/or semiautomatically manage the data being collected for storage in limited storage resources of the data management system. Thereby, the functionality of the data management system (e.g., to collect and store data for an individual) may be maintained without user input.

In an embodiment, a method for managing collection of data by a data management system and from data sources is disclosed. The method may include obtaining topics and topic rankings for the topics, the topics being relevant to a user for which the data management system provides data management services, and the topic rankings indicating relative levels of relevancy of each of the topics to the user; identifying a type of data that is relevant to the user using the topics and/or the topic rankings; identifying a portion of the data sources from which the type of data can be obtained; performing a selection process to obtain a management plan, the management plan defining actions to be performed by at least one data source of the portion of the data sources to obtain the type of the data for the data management system; and deploying the management plan to the at least one data source to initiate collection of the type of the data by the at least one data source to obtain collected data and distribution of the collected data to the data management system.

Each of the data sources may collect portions of collected data, and some of the portions of the collected data may include portions of the data managed by the data management system.

The method may further include a first data source of the data sources may be adapted to collect a first type of the collected data, a second data source of the data sources may be adapted to collect both the first type of the collected data and a second type of the collected data, and the first data source may be unable to collect the second type of the collected data.

The first type of the collected data may be a member of a topic of the topics, and the second type of the collected data may not be a member of any of the topics.

The first data source may include a first sensor adapted to measure a first property to obtain the first type of the collected data, and the second data source may include a second sensor adapted to measure a second property to obtain the second type of the collected data.

The first type of the collected data may be a member of a first topic of the topics, and the second type of the collected data may be a member of the first topic of the topics and a second topic of the topics.

The method may further include a first data source of the data sources may be adapted to collect a first type of the collected data, a second data source of the data sources may be adapted to collect a second type of the collected data, and the first data source may be unable to collect the second type of the collected data and the second data source may be unable to collect the first type of the collected data.

The topics and the topic rankings may be based at least in part on an audio transcript, the audio transcript may be based on an audio file, and the audio file may include audio data based on at least one conversation between two people.

The topics may include an enumeration of each unique topic of the topics discussed during the at least one conversation between the two people captured in the audio transcript; and the two people may include a first person for which the data is stored in the data management system, and a second person which provides at least one service to the first person.

Performing the selection process may include: identifying candidate data sources of the data sources that are each able to provide the type of the data; identifying, for each of the candidate data sources, limitations on other types of the data that is obtainable from the data sources; and using the limitations to identify at least one of the candidate data sources to collect the type of the data for the data management system.

The limitations may include different sensors adapted to measure at least one property to obtain different types of the collected data, the different types of data may include a first type of the collected data being a member of a first topic of the topics, and a second type of the collected data being a member of a second topic of the topics.

In an embodiment, a non-transitory media is provided. The non-transitory media may include instructions that when executed by a processor cause the computer-implemented method to be performed.

In an embodiment, a data processing system is provided. The data processing system may include the non-transitory media and a processor, and may perform the computer-implemented method when the computer instructions are executed by the processor.

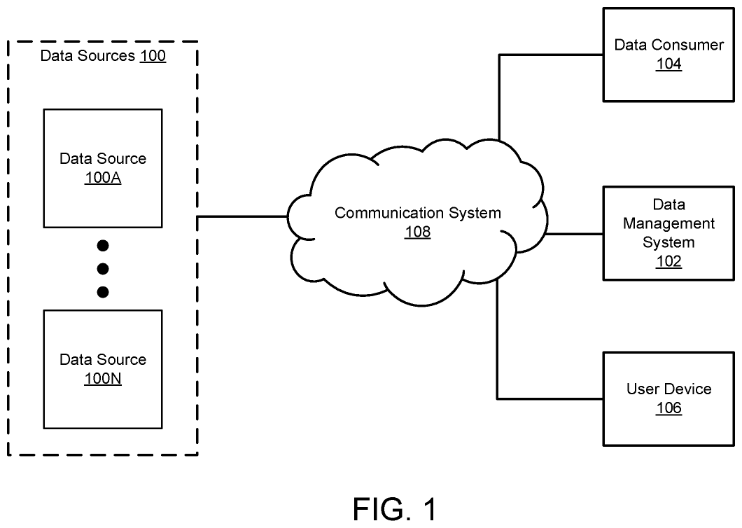

Turning to FIG. 1, a block diagram illustrating a system in accordance with an embodiment is shown. The system shown in FIG. 1 may provide computer-implemented services. The computer-implemented services may include data management services, data storage services, data access and control services, database services, and/or any other type of service that may be implemented with a computing device.

The system may include data management system 102. Data management system 102 may provide all, or a portion, of the computer-implemented services. To provide the computer-implemented services, data may be stored in data management system 102. The data stored in data management system 102 may include data usable (i) by an individual for which the data is stored, (ii) by other individuals to assist the individual, and/or (iii) by other individuals for other types of use. For example, the data may include healthcare information for an individual and the data may be usable by other individuals such as healthcare providers to diagnose and/or treat the individual for various health conditions.

The data stored in data management system 102 may be collected from data source 100. While illustrated with respect to a single data source, the system of FIG. 1 may include any number of data sources through which data management system 102 may obtain data. Data source 100 may include hardware and/or software components configured to obtain data, store data, provide data to other entities, and/or to perform any other task to facilitate performance of the computer-implemented services.

For example, an individual's healthcare information may be obtained from a healthcare provider system (e.g., data source 100) for use by the individual and/or other individuals (via associated devices). The data collected from data source 100 may include any quantity, size, and type of data. The data may include, for example, an audio recording (e.g., audio file) of a conversation between an individual and a healthcare provider, digitized results of medical tests, etc.

By storing data in data management system 102, the aggregated data may be usable for a variety of purposes. For example, in the healthcare context, the data may be usable for diagnostic purposes, verification purposes (e.g., second opinions), to facilitate studies by third parties that may use the data, etc. While described with respect to the healthcare services context, it will be appreciated that data may be stored in data management system 102 for other purposes and/or with respect to other contexts. For example, the stored data may be relevant for other types of services, uses, etc. without departing from embodiments disclosed herein.

However, some portions of the data stored in data management system 102 (on behalf of a user) may not include data relevant for use by the user and/or other individuals providing a service to the user. For example, in the healthcare context, a medical provider may require data for a patient relating to heart rate measurements in order to diagnose the patient. In some instances, the requested data (e.g., heart rate data) may not be included in the data stored in data management system 102. For example, data stored in data management system 102 may not include heart rate data for the patient if there was not a previous request for heart rate data and/or if a data source has not provided heart rate data for the patient.

In addition, some portions of the data collected and stored in data management system 102 may not be relevant for use by the individual for which the data is being stored and/or by other individuals for other types of use. Continuing the above example, the data collected by a data source (e.g., personal monitoring device such as a smart watch) for storage in data management system 102 may include heart rate measurements, blood pressure measurements, and sleep efficiency measurements for a patient. In this instance, the data relating to sleep efficiency for the user may not be relevant for purposes of diagnosing a potential condition related to high blood pressure such as hypertension (e.g., high blood pressure) by the medical provider. As such, collection and transmission of un-useful data by data source(s) for storage in data management system 102 may consume limited communication bandwidth of the data source(s) and storage resources available to data management system 102 which may be limited.

In general, embodiments disclosed herein may provide methods, systems, and/or devices for managing collection of data by data management systems from data sources. To manage collection of data, data management system 102 may limit the types of data being collected from data sources for storage in data management system 102. For example, data management system 102 may (i) identify a type of data relevant to a user, (ii) identify at least one data source able to obtain the relevant type of data, (iii) provide a request for the relevant type of data to the identified data source, and/or (iv) perform other types of data management actions with respect to various types of data managed by data management system 102.

Data management system 102 may select the types of data for performance of data management actions on the basis of relevancy of the types of data for one or more purposes. For example, some types of data collected, stored, and/or otherwise managed by data management system 102 may be more relevant or helpful for an individual and/or other individuals (e.g., service providers such as medical professional) to provide services to the individual than other types of data stored in the data management system. Collection of data that may include non-relevant information for an individual may be disadvantageous for the individual by reducing the ability of the desired services to be provided using the data managed by data management system 102 and/or by consuming limited storage resources of data management system. Therefore, data management system 102 may prioritize collection of data based on the relevancy of the data for one or more purposes with respect to an individual.

In order to discriminate more relevant data from less relevant data, the data management system may analyze the data being collected and stored, audio recordings of interactions between the individual and other individuals that provide services (e.g., a purpose for the data) to the individual, and/or other types of data that may include content relevant to discerning purposes (e.g., topics) that are relevant to the individual for which the data is stored. For example, data management system 102 may analyze an audio recording of a conversation between an individual and a healthcare provider to identify medical conditions impacting the individual. Based on this identification, data management system 102 may establish topics that are relevant to the individual, and prioritize collection and storage of data including and/or relating to the topics (e.g., in this example, diagnosis, treatment, etc. of these medical conditions) over collection and storage of data relating to other topics.

As new information regarding the topics becomes available, the topics and relevancy ratings (e.g., some topics may be of higher relevancy) for the topics may be updated. Consequently, the topics for which collection and storage of data is prioritized may be dynamically updated over time.

By dynamically updating the topics and relevancy rankings for the topics over time, embodiments disclosed herein may provide a collection system that is more likely to collect data that is more desirable to an individual, and not collect other data that is less desirable to the individual. The disclosed embodiments may do so in an automated and/or semiautomated fashion thereby reduce a cognitive burden on an individual for managing the data to be collected and stored in limited storage resources of data management system 102.

To provide the above noted functionality, the system of FIG. 1 may include data source 100, data management system 102, data consumer 104, user device 106, and communication system 108. Each of these components is discussed below.

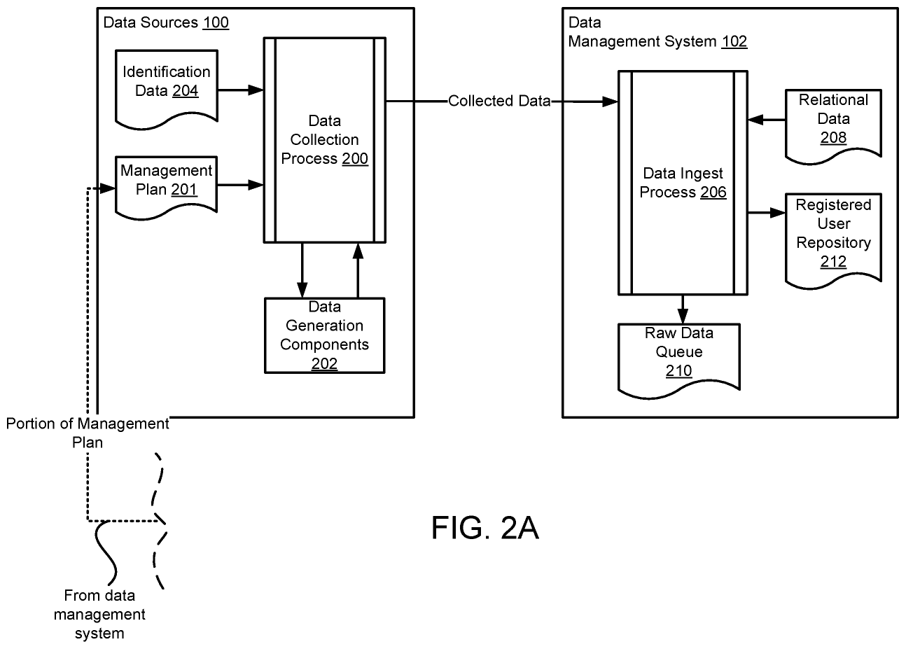

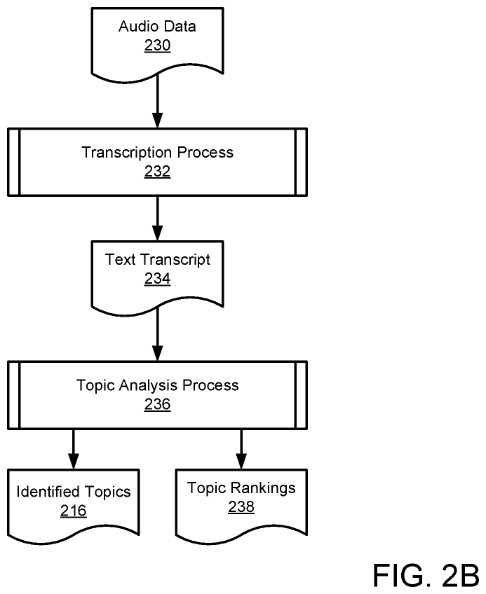

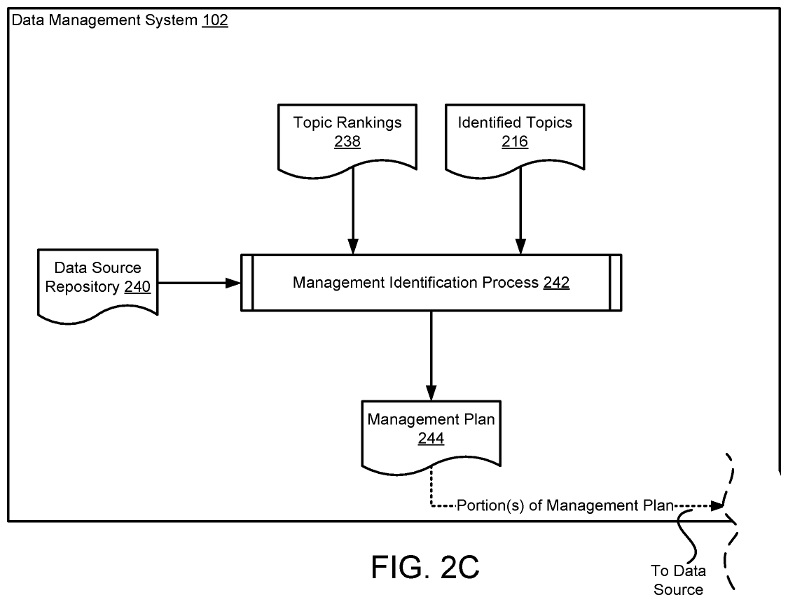

Data source 100 may (i) receive information relating to collection and transmission of data (e.g., specified by management plan 201 or a portion thereof) from data management system 102, (ii) facilitate collection and transmission of data (e.g., regarding and/or relating to an individual) to data management system 102, (iii) provide information identifying the individual or entity sourcing the data to data management system 102, and/or (vi) otherwise facilitate collection of data by data management system 102. Data source 100 may collect portions of collected data, and some of the portions of the collected data may include portions of the data managed by data management system 102. Data source 100 may be include a system operated by a medical provider which may collect, store, and/or provide access to data for a patient or individual, a personal device that collects information about an individual (e.g., cellphone, smart watch, etc.), and/or another type of data collection device. While described with respect to a medical provider, it will be appreciated that data source 100 may provide data related to other purposes without departing from embodiments disclosed herein. Refer to FIG. 2A for additional details regarding obtaining data using data source 100.

Data source 100 may be managed by (i) an individual or a patient for which the data is being collected, (ii) professional individuals that may provide a service for an individual, and/or (iii) other individuals or entities that may provide services for an individual. For example, data source 100 may be implemented using (i) a professional medical device and/or another device operated by a medical provider, (ii) personal electronic devices (e.g., personal monitoring devices such as smart watches and/or other wearable monitoring devices), and/or (iii) any other devices capable of collecting, storing, and/or providing data to data management system 102.

To manage collection of data, data management system 102 may (i) obtain data from data source 100, (ii) for audio data, perform a transcription process to obtain a text transcript of the audio data, (iii) perform an analysis of the text transcript of the data, (iv) based on the analysis of the text transcript, identify topics and topic rankings for the topics, (v) based on the topics and topic rankings, identify a type of data that is relevant to the user, (vi) identify data source(s) from which the type of data may be obtained, (vii) perform a selection process to obtain a management plan, (viii) selectively deploy the management plan based on the result of the selection process, and/or (xi) perform data management actions (e.g., based on topics and topic rankings for the user and/or other factors) to manage data collected by data management system 102. Similarly, data management system 102 may also provide access to stored data (e.g., to the individual for which the data is being managed and/or to data consumer 104). Refer to FIGS. 2A-2C for additional details regarding collecting data.

Data consumer 104 may (i) obtain limited access to selective portions of data stored in data management system 102, (ii) submit requests for access to data stored in data management system 102 by a third party or other individual, (iii) provide information identifying the individual or entity requesting access to the data and/or other types of information upon which decisions to grant access may be based, and/or (iv) once a request for access is granted (e.g., by user device 106), obtain access to data stored in data management system 102 (e.g., data for which access has been granted based on the submitted requests).

User device 106 may facilitate (i) access and control over data stored in data management system 102 by an individual, (ii) designation of portions of data for use by other individuals (e.g., data consumer 104), and/or (iii) performance of other management operations. User device 106 may be registered with data management system 102. For example, data management system 102 may confirm the identity of user device 106 based on a registration of the device, the registration may indicate that user device 106 is being used by the user or individual.

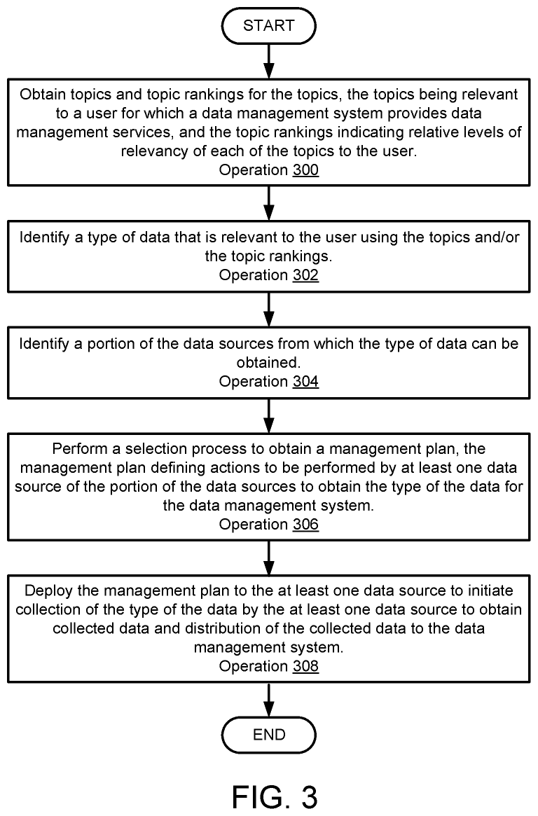

When providing their functionality, any of data source 100, data management system 102, data consumer 104, and/or user device 106 may perform all, or a portion, of the methods shown in FIG. 3.

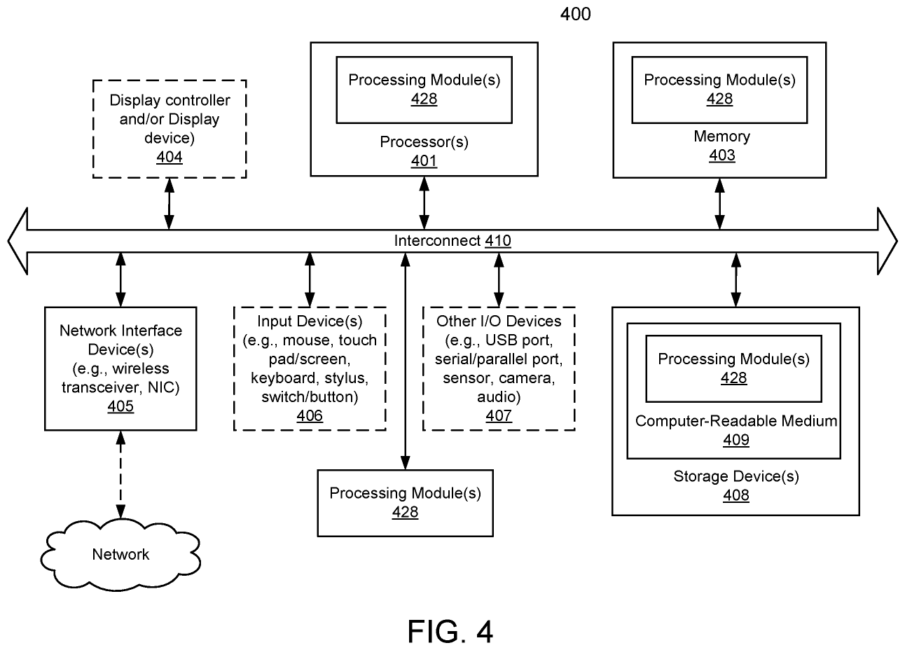

Any of (and/or components thereof) data source 100, data management system 102, data consumer 104, and user device 106 may be implemented using a computing device (also referred to as a data processing system) such as a host or a server, a personal computer (e.g., desktops, laptops, and tablets), a “thin” client, a personal digital assistant (PDA), a Web enabled appliance, a mobile phone (e.g., Smartphone), an embedded system, local controllers, an edge node, and/or any other type of data processing device or system. For additional details regarding computing devices, refer to FIG. 4.

Any of the components illustrated in FIG. 1 may be operably connected to each other (and/or components not illustrated) with communication system 108. In an embodiment, communication system 108 includes one or more networks that facilitate communication between any number of components. The networks may include wired networks and/or wireless networks (e.g., and/or the Internet). The networks may operate in accordance with any number and types of communication protocols (e.g., such as the internet protocol).

While illustrated in FIG. 1 as including a limited number of specific components, a system in accordance with an embodiment may include fewer, additional, and/or different components than those illustrated therein.

To further clarify embodiments disclosed herein, diagrams illustrating data flows implemented by a system over time in accordance with an embodiment are shown in FIGS. 2A-2C. In FIGS. 2A-2C, a first set of shapes (e.g., 204, 208) is used to represent data structures, a second set of shapes (e.g., 200, 206) is used to represent processes performed using data, and a third set of shapes (e.g., 202) is used to represent data generation components such as hardware components, software components, etc.

Turning to FIG. 2A, a first data flow diagram illustrating data flows, data processing, and/or other operations that may be performed by the system of FIG. 1 in accordance with an embodiment is shown. The data flows, data processing, and/or other operations may be performed when data is obtained from a data source. In FIG. 2A, example flows between data source 100 and data management system 102 are shown. It will be appreciated that similar data flow with respect to any devices (e.g., devices that may collect and transmit data to data management system 102 such as user device 106) and data management system 102 may be present.

To provide computer-implemented services, data management system 102 may obtain, store, and/or otherwise manage data for an individual. Data management system 102 may (i) obtain data from data source 100, and (ii) store some or all of the collected data for future use. However, data management system 102 may have a limited capacity for storing data. Consequently, data management system 102 may perform various data collection management processes over time, as discussed in greater detail with respect to FIGS. 2B-2C.

To obtain the data, data management system 102 and data sources 100 may cooperate with one another for data collection purposes.

To cooperate with data management system 102 for data collection purposes, data source 100 may perform data collection process 200. During data collection process 200, data may be (i) collected as specified by management plan 201, (ii) collected using data generation components 202, and (iii) provided all or a portion of the collected data (and/or derived data that is based at least in part on the collected data) to data management system 102.

Management plan 201 may include instructions for data source 100 (i) to collect a type of data relevant for use by a user and/or other individuals providing a service for the user, and (ii) provide the collected data to data management system 102. Refer to FIG. 2C for additional details regarding obtaining a management plan.

Data generation components 202 may include software components and/or hardware components to collect data. For example, data generation components 202 may include sensors, generative components, and display components of data source 100. The display components may be used to display prompts to a user of data source 100 (e.g., to instruct a user how to participate in data collection processes). The generative components may be used to generate various stimulations (e.g., optical, audio, etc.) for the user (e.g., so that data may be collected). The sensors may be used to obtain information regarding the user and the impact of the stimulations on the user.

Once collected, the data may be prepared for transmission to data management system. To prepare the collected data for transmission, the data may be enriched with additional information by adding metadata. The metadata may include, for example, (i) information regarding how the data was collected, (ii) information regarding for which entity the data was collected such as a user for which data management system 102 manages data, (iii) collection time, and/or other information that may enhance the collected data.

To add the metadata, data source 100 may store information regarding the user. For example, data source 100 may store identification data 204. Identification data 204 may include information regarding the identity of the individual for which the collected data is regarding/relating to. For example, identifying information such as the individual's name, date of birth, and/or any other identifying information for the individual for which the data is regarding.

Identification data 204 may also include information regarding the identity of the user and/or entity operating data source 100. For example, identifying information such as the user's and/or entity's name, IP address, and/or any other information useful to identify the operator and/or manager of data source 100.

Once enhanced, the collected data and corresponding metadata may be provided to data management system 102.

To cooperate with data source 100 for data collection purposes, data management system 102 may perform data ingest process 206. During data ingest process 206, the collected data obtained from data source 100 may be (i) classified with respect to which user the collected data is associated, (ii) managed in accordance with user-based access controls, and (iii) queued in raw data queue 210 for additional processing. Refer to FIGS. 2B-2C for additional details regarding the additional processing that may be performed on collected data.

To classify the data with respect to a user, the metadata may specify the user for which the data was collected. The user specified by the metadata may checked against users listed in registered user repository 212. Registered user repository 212 may include information regarding users that received data management services from data management system 102. Thus, when collected data is obtained, it may be verified as being relevant to users using registered user repository 212 (if not relevant, it may be discarded).

To manage the collected data in accordance with access controls, access to the data may be at least partially restricted. The restrictions for access to the collected data may be specified by relational data 208. Relational data 208 may specify restrictions on access to data managed by data management system 102 on behalf of different users. For example, the users may specify limits on the ability of other entities to access data managed by data management system 102 on behalf of the users.

For example, relational data 208 may specify whether and to what extent a data consumer (e.g., 104) may access the data stored by data management system 102 on behalf of a user. The access controls may be granular, thereby allowing a user to control which data consumers are able to access different portions of data. The access controls for a user may be established on a topic by topic basis. Thus, access to data for a given consumer may be given on a topic basis thereby allowing a user to provide a data consumer with access to all, or a portion, of the data managed by data management system that is related to one or more topics. Refer to FIGS. 2B-2C for additional information regarding topics.

To prepare the collected data for additional processing, the collected data may be queued in raw data queue 210. Raw data queue 210 may be implemented as a first in first out queue, or other type of queue. Raw data queue 210 may buffer data until it is processed and stored for long term retention.

Turning to FIG. 2B, a second data flow diagram illustrating data flows, data processing, and/or other operations that may be performed by the system of FIG. 1 in accordance with an embodiment is shown. The data flows, data processing, and/or other operations may be performed to identify topics relevant to a user, purpose, and/or another basis.

To obtain identified topics 216, data including clues and/or other information usable to identify topics that are relevant may be collected. For example, audio recordings of interactions (e.g., conversations) between an individual (e.g., a user of the data management system) and other individuals that provide services (e.g., a purpose for the data) to the individual may be obtained. The resulting audio data 230—and/or other types of data that may include content relevant to discern purposes (e.g., topics) relevant to the individual for which the data is being collected and stored—may be used to identify topics relevant to the individual.

For example, audio data 230 may include an audio recording of a conversation between a patient and a medical provider in which the two people discuss diagnosis, treatment, etc. for a particular type of medical condition such as diabetes. The conversation may be analyzed to identify topics (e.g., medical conditions, medical tests, etc.) that are relevant to the patient.

In order to analyze audio data 230, transcription process 232 may be performed. During transcription process 232, audio data 230 may be transcribed to obtain text transcript 234. Transcription process 232 may be performed using an inference model (not shown), artificial intelligence model (AI model), natural language processing, and/or automated transcription modalities. For example, audio data 230 may be ingested by an inference model through which audio data 230 is analyzed and transcribed into a text format (e.g., text transcript 234).

Once text transcript 234 is obtained, topic analysis process 236 may be performed in order to obtain identified topics 216 and topic rankings 238. Identified topics 216 may, as noted above, indicate topics that are relevant to a user of the data management system, and topic rankings 238 may indicate a rank order of the topics indicated by the identified topics 216. The rank order may be with respect to relevancy of the topics to the user.

During topic analysis process 236, text transcript 234 may be analyzed to (i) identify topics relevant to a user, and (ii) relative importance of each of the topics to the user.

To identify topics relevant to the user, text transcript 234 may be analyzed via (i) automated textual analysis to identify frequency/number of occurrences of difference utterances (e.g., words, phrases, etc.) made during the conversation captured in audio data 230, (ii) inferencing using inference models, (iii) large language model based natural language processing, and/or other text analysis modalities. The resulting output of any of these analyzation techniques may include a list of (i) topics that arose during the conversation captured in audio data 230, (ii) frequencies/counts of the topics, (iii) levels of emphasis on the different topics made by the different participants in the conversation, (iv) participants in the conversation that brought up the topics during the conversation, (v) duration of time during the conversation each topic was the topic of the conversation, (vi) opinion polarity (e.g., positive, neutral, negative, etc.) of each topic identified in the data, and/or other information regarding the topics during the conversation.

Identified topics 216 may be established based on any of the aforementioned information obtained via analysis of text transcript 234. For example, identified topics 216 may include (i) all topics that met a minimum threshold of interest (e.g., brought up above a threshold number of times/met a duration of time requirement as the topic of conversation) during the conversation captured by audio data 230, (ii) a prescribed number of the topics that were of the highest interest, etc.

Topic rankings 238 may be established based on the level of interest in each of identified topics 216 identified based on the conversation captured by audio data 230. For example, topics rankings 238 may rank identified topics 216 based on the number of times, frequency of utterance, and/or other quantification regarding interest in each of identified topics 216.

For example, an AI model may analyze text data (e.g., text transcript 234) regarding medical diagnosis, treatment, etc. for an individual and identify features (e.g., certain group of text or words) related to diabetes (e.g., topic). As such, the AI model may establish the topic of diabetes to be relevant to the individual and assign a relevancy value to the topic of diabetes (e.g., topic rankings 238).

Identified topics 216 and topic rankings 238 may be stored in a data repository (not shown) of data management system 102.

Over time, identified topics 216 and topics rankings 238 may be updated as new data is collected (e.g., audio data 230). Continuing with the above example, additional audio data that captures a conversation during which a new topic (e.g., such as a new medical condition) is discussed may be obtained and analyzed. Doing so may increase a relevancy value (e.g., topic ranking 238) for the new topic when compared to the topic of diabetes.

Once obtained, identified topics 216 and topics rankings 238 may be used to manage collection of data from one or more data sources by discriminating less relevant data from more relevant data in an automated manner.

Turning to FIG. 2C, a third data flow diagram illustrating data flows, data processing, and/or other operations that may be performed by the system of FIG. 1 in accordance with an embodiment is shown. The data flows, data processing, and/or other operations may be performed to manage collection of data by a data management system from data sources.

To manage collection of data, data management system 102 may perform management identification process 242. During management identification process 242, more relevant types of data may be discriminated from less relevant types of data stored in data management system 102 to identify a type of data (relevant to the user) to collect from a data source. For example, collection of data based on types of data relevant to a user may improve the ability of other individuals to provide services (e.g., medical diagnosis, treatment, etc.) which may be more relevant for the individual.

In addition, during management identification process 242, the capabilities and/or limitations of data sources (e.g., obtained via data source repository 240) may be analyzed to select at least one data source to collect the identified type of data for the user and to distribute to data management system 102. For example, selecting a data source capable of obtaining a single type of data (e.g., temperature data) when the type of data identified is related to temperature data may improve the likelihood that another data source capable of obtaining two types of data (e.g., temperature and/or heart rate data) may be available for use when the type of data identified is related to heart rate data.

The identified type of data relevant to a user and the selected data source(s) may be used to obtain management plan 244. Management plan 244 may include instructions for actions to be performed by at least one data source to obtain the type of the data for data management system 102.

To identify a type of data relevant to a user, data management system 102 may use identified topics 216 and topic rankings 238 during management identification process 242. Identified topics 216, as noted above, may specify topics that have been identified as being relevant to an individual and topic rankings 238 may include a rank ordering of the topics (e.g., identified topics 216). Identified topics 216 and topic rankings 238 may be obtained from a data repository (not shown) within data management system 102.

Once obtained, a type of data relevant to the user may be identified by performing any type of classification processes using, for example, inference models (e.g., decision trees, machine learning models, rules based systems, etc.). In some instances, at least some inference models are implemented by training a neural network to perform classification. The neural network may be trained using supervised learning, self-supervised learning, semi-supervised learning, and/or unsupervised learning. For example, with supervised learning, some number of instances of data may be hand-labeled by a subject matter expert or other person with respect to the topics (may be any number of topics, may include more topics than identified topics 216) for which the data is relevant to obtain a training data set. Once obtained, the training data set may be used to train the neural network (e.g., to set the weights of neurons and/or other features of the neural network).

In an embodiment, an inference model may be trained to identify different types of data based on topics (e.g., identified topics 216). For example, an inference model may specify a range of different topics to which a type of data is relevant. The range of different topics may include a variety of topics that may be relevant to the individual and/or user for which the data is being stored in data management system 102.

To identify a type of data that is relevant to the user, identified topics 216 and topic rankings 238 may be ingested by an inference model. In this instance, the inference model may, as output, indicate a type of data that is relevant to the user. For example, if identified topics 216 specify the topics of “headaches”, “chills”, and “fever” as the higher ranked topics (e.g., via topic rankings 238), then the inference model may identify “temperature data” as the type of data that is relevant to the user (e.g., inference model may link the topics to a type of data that needs to be collected). In this health-care related example, the output of the type of data (e.g., temperature data) may be used to collect necessary data in order for a medical professional to provide medical care (e.g., diagnosis, treatment, etc.) for the user.

Once identified, the type of data may be used in order to identify a portion of data sources (e.g., potential and/or candidate data sources) from which the type of data may be obtained. The portion of data sources may be any data sources identified and/or information regarding the data sources stored in data source repository 240. Data source repository 240 may include information regarding various data sources available to collect data for a user for which data management system 102 may provide data management services.

In an embodiment, each of the data sources (e.g., identified in data source repository 240) may collect, at least, one type of data. In some instances, the capabilities of each data source (e.g., from data source repository 240) to collect different types of data may be based on the components of each respective data source. For example, data source A may include a sensor (e.g., inertial measurement unit sensor) adapted to measure body movement of a user and data source B may include a sensor (e.g., temperature sensor) adapted to measure body temperature of the user.

In some instances, continuing the above example, the first data source (data source A) of the data sources may be adapted to collect a first type of the collected data (body movement data) and the second data source (data source B) may be adapted to collect a second type of the collected data (body temperature data). In this instance, the first data source (data source A) may be unable to collect the second type of the collected data (body temperature data) and the second data source (data source B) may be unable to collect the first type of the collected data (body movement data).

In some instances, a portion the data sources may be capable of collecting multiple types of data. For example, data source C may include sensor(s) (e.g., inertial measurement unit sensors, heart rate sensors, etc.) adapted to measure body movement and heart rate of a user. Therefore, each data source included in data source repository 240 may be identified based on the type(s) of data that the data source may obtain.

During management identification process 242, the type of data identified as relevant to the user (e.g., an identifier for the type of data) may be used in performing a look up and/or any other parsing process to identify potential and/or candidate data sources from data source repository 240. In some instances, the data sources (e.g., including information related to capabilities and/or limitations of the data sources) may be stored in a searchable format keyed to different types of data in data source repository 240. Continuing the above example, an identifier for body movement data (e.g., the type of data) may be used as a key to identify data source A and data source C (e.g., data sources capable of collecting body movement data) as the portion of data sources (e.g., potential and/or candidate data sources).

In an embodiment, management identification process 242 may include performing a selection process to obtain a management plan (e.g., management plan 244). The selection process may include analyzing the portion of data sources (e.g., capabilities, limitations, etc.) to identify at least one data source of the portion of data sources to collect the type of data. For example, the selection process may be facilitated by data management system 102 by (i) identifying candidate data sources of the data sources that are each able to provide the type of data, (ii) identifying, for each of the candidate data sources, limitations on other types of data that is obtainable from the data sources, and/or (iii) using the limitations to identify at least one of the candidate data sources to collect the type of the data for data management system 102.

In some instances, the limitations for each data source may include different sensors adapted to measure at least one property to obtain different types of the collected data. For example, the different types of data may include a first type of the collected data (e.g., heart rate data) being a member of a first topic (e.g., heart condition) of the topics (e.g., identified topics 216), and a second type of the collected data (e.g., blood pressure levels) being a member of a second topic (e.g., cardiovascular condition) of the topics (e.g., identified topics 216).

By performing the selection process described above, the type of data relevant to the user may be collected by a data source while improving the usability of other data sources capable of obtaining other types of data. Continuing the above example, data management system 102 may identify data source A and data source C as the candidate data sources based on “body movement data” as the identified type of data relevant to the user. Data source A may be adapted to collect heart rate data and body movement data and data source C may be adapted to collect body movement data. In this instance, data management system 102 may select data source C to collect the body movement data for the user based on the limitations of data source A to collect other types of data. Thus, data source A may be available to collect heart rate data at the time that type of data is identified as relevant to the user.

Information including the type of data, the selected data source, and any other information obtained through management identification process 242 may be included in management plan 244. For example, management plan 244 may include instructions for deployment, management, and/or collection of the type of data (e.g., relevant to the user) by the selected data source(s) (e.g., at least one data source) and distribution of the collected data to data management system 102.

Continuing the above example, management plan 244 may include instructions for data source C to collect body movement data (e.g., type of data) for a period of time and distribute the collected body movement data to data management system 102.

In some instances, management plan 244 may indicate a preferential ranking of the candidate data sources capable of obtaining the type of data relevant to the user. In some instances, the data source selected to collect the type of data may be based on the preferential ranking in order to reduce an instance of the type of data not being collected for distribution to data management system 102 and maximize the resources of the other data sources. Continuing the above example, management plan 244 may indicate data source C to be the most likely of the data sources to collect body movement data (e.g., type of data) and to provide the collected data to data management system 102 while maximizing the capabilities of the other data source (e.g., data source A). In some instances, if data source C is unable to collect the type of data for some reason after a period of time then data source A may be instructed to collect the type of data for distribution to data management system 102.

Management plan 244 may be provided to at least one data source to initiate collection of the type of the data by the at least one data source to obtain collected data and distribution of the collected data to data management system 102. In some instances, the management plan (e.g., 244) may be deployed to the data source specified by the management plan (e.g., 244) to obtain the type of data identified. For example, data management system 102 may select data source A from the data sources to obtain the type of data for distribution to data management system 102. As such, data management system 102 may deploy management plan 244 to data source A to initiate collection of the data.

In an embodiment, data management system 102 may deploy management plan 244 (and/or a portion thereof) to multiple data sources such that collection of the type of the data may be initiated by at least one of the data sources specified by management plan 244. By doing so, the data sources may collectively receive management plan 244 to obtain collected data as a result. For example, data management system 102 may deploy copies of the management plan (e.g., 244) to one or more of the data sources (e.g., data source 100) to increase the likelihood that execution of management plan 244 may be performed by at least one of the data sources capable of obtaining the type of data for distribution to data management system 102.

In some embodiments, data management system 102 may manage the deployment of management plan 244. During deployment of management plan 244, at least one of data sources may be unable to complete execution of management plan 244. Managing execution of management plan 244 may be based on the management plan (e.g., 244) and may include, at least in part, deployment of additional copies of management plan 244 to the other data sources (e.g., capable of obtaining the type of data relevant to the user) to provide eventual compliance with the management plan (e.g., 244) through which the collected data is obtained and distributed to data management system 102. The collected data obtained and distributed to data management system 102 may be used by the user and/or other individuals, such as data consumers (e.g., 104) for providing a service to the user.

Thus, using the data flows and processes shown in FIGS. 2A-2C, collection of data may be automatically managed via discrimination of less relevant types of data from more relevant types of data and selection of data source(s) to obtain relevant types of data based on comparison of the capabilities and/or limitations of data sources. The discriminated more relevant data may be utilized to manage what types of data is to be collected and which of the data sources to provide the types of data.

As discussed above, the components of FIGS. 1-2C may perform various methods to manage operation of data processing systems. FIG. 3 illustrates a method that may be performed by the components of the system of FIGS. 1-2C. In the diagram discussed below and shown in FIG. 3, any of the operations may be repeated, performed in different orders, omitted, and/or performed in parallel with or in a partially overlapping in time manner with other operations.

Turning to FIG. 3, a flow diagram illustrating a method for managing collection of data by a data management system in accordance with an embodiment is shown. The method may be performed, for example, by any of data source 100, data management system 102, data consumer 104, user device 106, and/or other components of the system shown in FIGS. 1-2C.

Prior to operation 300, a data management system may have obtained data for an individual and stored the data in a data repository within the data management system. The data may have been obtained through various processes such as generation, acquisition from external entity (e.g., medical provider, monitoring devices, etc.), acquisition from the individual whose data is being stored, and/or by any other method. The data may include data relating to healthcare information for an individual (e.g., medical records) and/or topics discussed during conversations between a first person and a second person. The data may be classified and processed by the data management system based on topics (e.g., types of data) relevant to the individual. To initiate collection of data relevant to the user, the data management system may obtain topics and topic rankings for the topics for an individual.

At operation 300, topics and topic rankings for the topics may be obtained. The topics and topic rankings for the topics may be obtained by (i) receiving information regarding topics and topic rankings, (ii) generating the topics and topic rankings, and/or (iii) via any other method to obtain topics and topic rankings. For example, the topics and the topic rankings may be generated by (i) obtaining an audio transcript from a data source, (ii) performing a transcription process, using the audio transcript, to obtain a text transcript, (iii) performing an analysis of the text transcript to identify topics and topic rankings for the topics.

The topics may be relevant to a user for which a data management system provides data management services. The topics for the data may, as discussed above, be based at least in part on the topics discussed during a conversation between two people. The topics may include an enumeration of each unique topic of topics discussed during the conversation between the two people. For example, an audio recording may include a conversation between patient and a medical provider discussing a patient's diabetes diagnosis, treatment, etc. In this example, diabetes may be identified as the topic in which the collected data from a data source (e.g., a personal monitoring device such as a smart watch) may be associated.

The topic rankings may be based, at least in part, on instances of the topics discussed during a conversation between the two people. For example, a counter of the utterances for the topics, duration of conversation dedicated to each topic, and/or other quantifications may be derived from the conversation. The topic rankings may be based on these quantifications (e.g., more frequently uttered topics may be ranked more highly than less frequently uttered topics). The topic rankings may indicate, at least in part, relative levels of relevancy of each of the topics to the user.

At operation 302, a type of data that is relevant to the user may be identified using the topics and/or the topic rankings. The type of data may be identified by (i) rank ordering the topics based on the topic rankings, (ii) selecting, based on the rank ordering of the topics, the topic most relevant to the user, and/or (iii) any other method.

The type of data may be identified (i) by providing the topics and topic rankings to a third party and/or entity to perform a classification process, (ii) via generation by the data management system, and/or (iii) by performing any other methods. Identifying the type of data via generation may include performing a classification process of the topics and topic rankings using an inference model trained to identify types of data based on topics identified as being relevant to the user. The type of data may be used to identify a portion of the data sources from which the type of data may be obtained.

At operation 304, a portion of the data sources from which the type of data can be obtained may be identified. The portion of the data sources may be identified by (i) obtaining a list of data sources from data source repository, (ii) making a comparison, using an identifier for the identified type of data, between the type of data and the data sources capable of obtaining the type of data, and/or (iii) any other methods. The portion of the data sources may include any data sources capable of obtaining at least the type of data identified via operation 302 and/or a combination of the type of data identified via operation 302 and other types of data.

At operation 306, a selection process may be performed to obtain a management plan. The management plan may define actions to be performed by at least one data source of the portion of the data sources to obtain the type of the data for the data management system. The selection process may be performed by (i) identifying candidate data sources of the data sources that are each able to provide the type of the data, (ii) identifying, for each of the candidate data sources, limitations on other types of the data that is obtainable from the data sources, and/or (iii) using the limitations to identify at least one of the candidate data sources to collect the type of the data for the data management system.

Identifying candidate data sources of the data sources may be performed by (i) receiving it from a third party and/or entity via communication by a data processing system, (ii) reading it from storage (e.g., data source repository), and/or (iii) via any other method. Candidate data sources of the data sources may include any data sources capable of providing the type of data identified via operation 302.

Identifying, for each of the candidate data sources, limitations on other types of the data may be performed by (i) receiving the limitations from a third party and/or entity via communication by a data processing system, (ii) reading the limitations from storage (e.g., data source repository), and/or (iii) via any other method. The limitations on other types of the data that may be obtainable from the data sources may include different sensors adapted to measure at least one property to obtain different types of the collected data. For example, data source 100A may be capable of obtaining a first type of data (e.g., heart rate data) which may be one of the topics identified (e.g., identified topics 216) and data source 100N may be capable of obtaining a second type of data (e.g., sleep cycle data) which may not be one of the topics identified for the user.

Identifying at least one of the candidate data sources using the limitations may be performed by making a comparison between the candidate data sources and the identified limitations on other types of the data. For example, data source 100A may be limited to collecting one type of data (e.g., blood sugar data) at a time and data source 100N may be capable of collecting two types of data (e.g., blood sugar data and/or heart rate data) during a single period of time. As such, data management system 102 may decide, based on the limitation of collecting one type of data by data source 100A, to select data source 100A as the data source to initiate collection of the type of data. Doing so may increase the availability of data sources (e.g., data source 100N) to collect and distribute other types of data relevant to the user (such as heart rate data).

Some data sources (e.g., data source 100) may have limitations that affect the ability of the data source to collect specific types of data. These limitations may be based on the different sensors which may adapt to measure different types of the collected data. When selecting which data source to collect the type of data relevant to the user, data management system 102 may select the data source with a limitation to collect only the type of data identified as the most relevant during management identification process 242.

For example, the identified type of data may be heart rate data and a first data source (e.g., data source A) may be capable of collecting heart rate data and a second data source (e.g., data source B) may be capable of collecting both heart rate data and blood sugar data. In this instance, data management system 102 may select data source A to initiate collection of heart rate data (e.g., identified type of data relevant to the user) due to the limitation of data source B to collect different types of data. As such, data source B may be available to collect other types of data when identified by data management system 102 (e.g., during management identification process 242).

At operation 308, the management plan may be deployed to the at least one data source to initiate collection of the type of the data by the at least one data source to obtain collected data and distribution of the collected data to the data management system. The management plan may be deployed by (i) providing the management plan to the at least one data source via electronic communication from the data management system, (ii) providing the management plan to a third-party entity and/or operating system to provide to at least one data source, and/or (iii) any other methods.

The method may end following operation 308.

Using the methods illustrated in FIG. 3, embodiments disclosed herein may facilitate collection of data by a data management in which data is stored on behalf of an individual. Collection of data may include prioritizing the type of data and at least one data source capable of obtaining the type of data relevant to the user.

Any of the components illustrated in FIGS. 1-2C may be implemented with one or more computing devices. Turning to FIG. 4, a block diagram illustrating an example of a data processing system (e.g., a computing device) in accordance with an embodiment is shown. For example, system 400 may represent any of data processing systems described above performing any of the processes or methods described above. System 400 can include many different components. These components can be implemented as integrated circuits (ICs), portions thereof, discrete electronic devices, or other modules adapted to a circuit board such as a motherboard or add-in card of the computer system, or as components otherwise incorporated within a chassis of the computer system. Note also that system 400 is intended to show a high level view of many components of the computer system. However, it is to be understood that additional components may be present in certain implementations and furthermore, different arrangement of the components shown may occur in other implementations. System 400 may represent a desktop, a laptop, a tablet, a server, a mobile phone, a media player, a personal digital assistant (PDA), a personal communicator, a gaming device, a network router or hub, a wireless access point (AP) or repeater, a set-top box, or a combination thereof. Further, while only a single machine or system is illustrated, the term “machine” or “system” shall also be taken to include any collection of machines or systems that individually or jointly execute a set (or multiple sets) of instructions to perform any one or more of the methodologies discussed herein.

In one embodiment, system 400 includes processor 401, memory 403, and devices 405-407 via a bus or an interconnect 410. Processor 401 may represent a single processor or multiple processors with a single processor core or multiple processor cores included therein. Processor 401 may represent one or more general-purpose processors such as a microprocessor, a central processing unit (CPU), or the like. More particularly, processor 401 may be a complex instruction set computing (CISC) microprocessor, reduced instruction set computing (RISC) microprocessor, very long instruction word (VLIW) microprocessor, or processor implementing other instruction sets, or processors implementing a combination of instruction sets. Processor 401 may also be one or more special-purpose processors such as an application specific integrated circuit (ASIC), a cellular or baseband processor, a field programmable gate array (FPGA), a digital signal processor (DSP), a network processor, a graphics processor, a network processor, a communications processor, a cryptographic processor, a co-processor, an embedded processor, or any other type of logic capable of processing instructions.

Processor 401, which may be a low power multi-core processor socket such as an ultra-low voltage processor, may act as a main processing unit and central hub for communication with the various components of the system. Such processor can be implemented as a system on chip (SoC). Processor 401 is configured to execute instructions for performing the operations discussed herein. System 400 may further include a graphics interface that communicates with optional graphics subsystem 404, which may include a display controller, a graphics processor, and/or a display device.

Processor 401 may communicate with memory 403, which in one embodiment can be implemented via multiple memory devices to provide for a given amount of system memory. Memory 403 may include one or more volatile storage (or memory) devices such as random access memory (RAM), dynamic RAM (DRAM), synchronous DRAM (SDRAM), static RAM (SRAM), or other types of storage devices. Memory 403 may store information including sequences of instructions that are executed by processor 401, or any other device. For example, executable code and/or data of a variety of operating systems, device drivers, firmware (e.g., input output basic system or BIOS), and/or applications can be loaded in memory 403 and executed by processor 401. An operating system can be any kind of operating systems, such as, for example, Windows® operating system from Microsoft®, Mac OS®/iOS® from Apple, Android® from Google®, Linux®, Unix®, or other real-time or embedded operating systems such as VxWorks.

System 400 may further include IO devices such as devices (e.g., 405, 406, 407, 408) including network interface device(s) 405, optional input device(s) 406, and other optional IO device(s) 407. Network interface device(s) 405 may include a wireless transceiver and/or a network interface card (NIC). The wireless transceiver may be a WiFi transceiver, an infrared transceiver, a Bluetooth transceiver, a WiMax transceiver, a wireless cellular telephony transceiver, a satellite transceiver (e.g., a global positioning system (GPS) transceiver), or other radio frequency (RF) transceivers, or a combination thereof. The NIC may be an Ethernet card.

Input device(s) 406 may include a mouse, a touch pad, a touch sensitive screen (which may be integrated with a display device of optional graphics subsystem 404), a pointer device such as a stylus, and/or a keyboard (e.g., physical keyboard or a virtual keyboard displayed as part of a touch sensitive screen). For example, input device(s) 406 may include a touch screen controller coupled to a touch screen. The touch screen and touch screen controller can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with the touch screen.

IO devices 407 may include an audio device. An audio device may include a speaker and/or a microphone to facilitate voice-enabled functions, such as voice recognition, voice replication, digital recording, and/or telephony functions. Other IO devices 407 may further include universal serial bus (USB) port(s), parallel port(s), serial port(s), a printer, a network interface, a bus bridge (e.g., a PCI-PCI bridge), sensor(s) (e.g., a motion sensor such as an accelerometer, gyroscope, a magnetometer, a light sensor, compass, a proximity sensor, etc.), or a combination thereof. IO device(s) 407 may further include an imaging processing subsystem (e.g., a camera), which may include an optical sensor, such as a charged coupled device (CCD) or a complementary metal-oxide semiconductor (CMOS) optical sensor, utilized to facilitate camera functions, such as recording photographs and video clips. Certain sensors may be coupled to interconnect 410 via a sensor hub (not shown), while other devices such as a keyboard or thermal sensor may be controlled by an embedded controller (not shown), dependent upon the specific configuration or design of system 400.

To provide for persistent storage of information such as data, applications, one or more operating systems and so forth, a mass storage (not shown) may also couple to processor 401. In various embodiments, to enable a thinner and lighter system design as well as to improve system responsiveness, this mass storage may be implemented via a solid state device (SSD). However, in other embodiments, the mass storage may primarily be implemented using a hard disk drive (HDD) with a smaller amount of SSD storage to act as an SSD cache to enable non-volatile storage of context state and other such information during power down events so that a fast power up can occur on re-initiation of system activities. Also a flash device may be coupled to processor 401, e.g., via a serial peripheral interface (SPI). This flash device may provide for non-volatile storage of system software, including a basic input/output software (BIOS) as well as other firmware of the system.

Storage device 408 may include computer-readable storage medium 409 (also known as a machine-readable storage medium or a computer-readable medium) on which is stored one or more sets of instructions or software (e.g., processing module, unit, and/or processing module/unit/logic 428) embodying any one or more of the methodologies or functions described herein. Processing module/unit/logic 428 may represent any of the components described above. Processing module/unit/logic 428 may also reside, completely or at least partially, within memory 403 and/or within processor 401 during execution thereof by system 400, memory 403 and processor 401 also constituting machine-accessible storage media. Processing module/unit/logic 428 may further be transmitted or received over a network via network interface device(s) 405.

Computer-readable storage medium 409 may also be used to store some software functionalities described above persistently. While computer-readable storage medium 409 is shown in an exemplary embodiment to be a single medium, the term “computer-readable storage medium” should be taken to include a single medium or multiple media (e.g., a centralized or distributed database, and/or associated caches and servers) that store the one or more sets of instructions. The terms “computer-readable storage medium” shall also be taken to include any medium that is capable of storing or encoding a set of instructions for execution by the machine and that cause the machine to perform any one or more of the methodologies of embodiments disclosed herein. The term “computer-readable storage medium” shall accordingly be taken to include, but not be limited to, solid-state memories, and optical and magnetic media, or any other non-transitory machine-readable medium.

Processing module/unit/logic 428, components and other features described herein can be implemented as discrete hardware components or integrated in the functionality of hardware components such as ASICS, FPGAs, DSPs or similar devices. In addition, processing module/unit/logic 428 can be implemented as firmware or functional circuitry within hardware devices. Further, processing module/unit/logic 428 can be implemented in any combination hardware devices and software components.

Note that while system 400 is illustrated with various components of a data processing system, it is not intended to represent any particular architecture or manner of interconnecting the components; as such details are not germane to embodiments disclosed herein. It will also be appreciated that network computers, handheld computers, mobile phones, servers, and/or other data processing systems which have fewer components or perhaps more components may also be used with embodiments disclosed herein.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Embodiments disclosed herein also relate to an apparatus for performing the operations herein. Such a computer program is stored in a non-transitory computer readable medium. A non-transitory machine-readable medium includes any mechanism for storing information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices).

The processes or methods depicted in the preceding figures may be performed by processing logic that comprises hardware (e.g. circuitry, dedicated logic, etc.), software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

Embodiments disclosed herein are not described with reference to any particular programming language. It will be appreciated that a variety of programming languages may be used to implement the teachings of embodiments disclosed herein.

In the foregoing specification, embodiments have been described with reference to specific exemplary embodiments thereof. It will be evident that various modifications may be made thereto without departing from the broader spirit and scope of the embodiments disclosed herein as set forth in the following claims. The specification and drawings are, accordingly, to be regarded in an illustrative sense rather than a restrictive sense.

1. A method for managing collection of data by a data management system and from data sources, the method comprising:
obtaining, by the data management system, topics and topic rankings for the topics, the topics being relevant to a user for which the data management system provides data management services, and the topic rankings indicating relative levels of relevancy of each of the topics to the user;
identifying, by the data management system, a type of data that is relevant to the user using the topics and/or the topic rankings;
identifying, by the data management system, a portion of the data sources from which the type of data can be obtained;
performing, by the data management system, a selection process to obtain a management plan, the management plan defining actions to be performed by at least one data source of the portion of the data sources to obtain the type of the data for the data management system; and
transmitting, by the data management system and to the at least one data source, the management plan to cause the at least one data source to collect the type of the data as collected data and distribute the collected data to the data management system, the data management system in turn provides the collected data to a user device associated with a user.

2. The method of claim 1, wherein the management plan is provided, by the data management system, to each of the data sources to cause each of the data sources to collect portions of the collected data, and some of the portions of the collected data comprise data already stored in the data management system before the collected data is provided to the data management system.

3. The method of claim 2, wherein a first data source of the data sources is adapted to collect a first type of the collected data, a second data source of the data sources is adapted to collect both the first type of the collected data and a second type of the collected data, and the first data source is unable to collect the second type of the collected data.

4. The method of claim 3, wherein the first type of the collected data is a member of a topic of the topics, and the second type of the collected data is not a member of any of the topics.

5. The method of claim 4, wherein the first data source comprises a first sensor adapted to measure a first property to obtain the first type of the collected data, and the second data source comprises a second sensor adapted to measure a second property to obtain the second type of the collected data.

6. The method of claim 3, wherein the first type of the collected data is a member of a first topic of the topics, and the second type of the collected data is a member of the first topic of the topics and a second topic of the topics.

7. The method of claim 2, wherein a first data source of the data sources is adapted to collect a first type of the collected data, a second data source of the data sources is adapted to collect a second type of the collected data, and the first data source is unable to collect the second type of the collected data and the second data source is unable to collect the first type of the collected data.

8. The method of claim 1, wherein the topics and the topic rankings are based at least in part on an audio transcript, the audio transcript being based on an audio file, and the audio file comprising audio data based on at least one conversation between two people.

9. The method of claim 8, wherein the topics comprise an enumeration of each unique topic of the topics discussed during the at least one conversation between the two people captured in the audio transcript; and the two people comprise a first person for which the data is stored in the data management system, and a second person which provides at least one service to the first person.

10. The method of claim 1, wherein performing the selection process comprises:
identifying candidate data sources of the data sources that are each able to provide the type of the data;
identifying, for each of the candidate data sources, limitations on other types of the data that is obtainable from the data sources; and
using the limitations to identify at least one of the candidate data sources to collect the type of the data for the data management system.

11. The method of claim 10, where the limitations comprise different sensors adapted to measure at least one property to obtain different types of the collected data, the different types of data comprising a first type of the collected data being a member of a first topic of the topics, and a second type of the collected data being a member of a second topic of the topics.

12. A non-transitory machine-readable medium having instructions stored therein, which when executed by a processor of a data management system, cause the processor to perform operations for managing collection of data by the data management system and from data sources, the operations comprising:
obtaining, by the data management system, topics and topic rankings for the topics, the topics being relevant to a user for which the data management system provides data management services, and the topic rankings indicating relative levels of relevancy of each of the topics to the user;
identifying, by the data management system, a type of data that is relevant to the user using the topics and/or the topic rankings;
identifying, by the data management system, a portion of the data sources from which the type of data can be obtained;
performing, by the data management system, a selection process to obtain a management plan, the management plan defining actions to be performed by at least one data source of the portion of the data sources to obtain the type of the data for the data management system; and
transmitting, by the data management system and to the at least one data source, the management plan to cause the at least one data source to collect the type of the data as collected data and distribute the collected data to the data management system, the data management system in turn provides the collected data to a user device associated with a user.

13. The non-transitory machine-readable medium of claim 12, wherein the management plan is provided, by the data management system, to each of the data sources to cause each of the data sources to collect portions of the collected data, and some of the portions of the collected data comprise data already stored in the data management system before the collected data is provided to the data management system.

14. The non-transitory machine-readable medium of claim 13, wherein a first data source of the data sources is adapted to collect a first type of the collected data, a second data source of the data sources is adapted to collect both the first type of the collected data and a second type of the collected data, and the first data source is unable to collect the second type of the collected data.

15. The non-transitory machine-readable medium of claim 14, wherein the first type of the collected data is a member of a topic of the topics, and the second type of the collected data is not a member of any of the topics.

16. The non-transitory machine-readable medium of claim 15, wherein the first data source comprises a first sensor adapted to measure a first property to obtain the first type of the collected data, and the second data source comprises a second sensor adapted to measure a second property to obtain the second type of the collected data.

17. A data management system, comprising:
a processor; and
a memory coupled to the processor to store instructions, which when executed by the processor, cause the processor to perform operations for managing collection of data by the data management system and from data sources, the operations comprising:
obtaining, by the data management system, topics and topic rankings for the topics, the topics being relevant to a user for which the data management system provides data management services, and the topic rankings indicating relative levels of relevancy of each of the topics to the user;
identifying, by the data management system, a type of data that is relevant to the user using the topics and/or the topic rankings;
identifying, by the data management system, a portion of the data sources from which the type of data can be obtained;
performing, by the data management system, a selection process to obtain a management plan, the management plan defining actions to be performed by at least one data source of the portion of the data sources to obtain the type of the data for the data management system; and
transmitting, by the data management system and to the at least one data source, the management plan to cause the at least one data source to collect the type of the data as collected data and distribute the collected data to the data management system, the data management system in turn provides the collected data to a user device associated with a user.

18. The data processing system of claim 17, wherein the management plan is provided, by the data management system, to each of the data sources to cause each of the data sources to collect portions of the collected data, and some of the portions of the collected data comprise data already stored in the data management system before the collected data is provided to the data management system.

19. The data processing system of claim 18, wherein a first data source of the data sources is adapted to collect a first type of the collected data, a second data source of the data sources is adapted to collect both the first type of the collected data and a second type of the collected data, and the first data source is unable to collect the second type of the collected data.

20. The data processing system of claim 19, wherein the first type of the collected data is a member of a topic of the topics, and the second type of the collected data is not a member of any of the topics.
